(19) United States
(12) Patent Application Publication
(10) Pub. No.: US 2006/0079677 A1
(43) Pub. Date: Apr. 13, 2006

(54) Novel tricyclic azepine derivatives, method for production thereof and pharmaceutical compositions comprising the same

(76) Inventors: Sebastien Gallet, Fouquereuil (FR); Pascal Berthelot, Haubourdin (FR); Nicolas Lebegue, Wignehies (FR); Nathalie Flouquet, Marquillies (FR); Pascal Carato, Lille (FR); John Hickman, Paris (FR); Alain Pierre, Les Alluets Le Roi (FR); Bruno Pfeiffer, Saint Leu La Foret (FR); Pierre Renard, Le Chesnay (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/543,729

(22) PCT Filed: Feb. 3, 2004
(86) PCT No.: PCT/FR04/00234

§ 371 Date: Jul. 26, 2005

(30) Foreign Application Priority Data
Feb. 3, 2003 (FR) 03/01181

(51) Int. Cl. C07D 498/02; C07D 487/02

(52) U.S. Cl. 540/497; 544/14; 540/548

(57) ABSTRACT

A compound of formula (I): 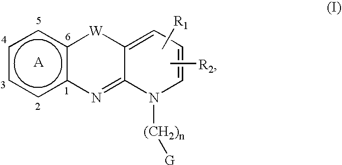
wherein: 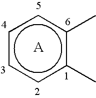
represents benzo or pyrido, optionally fused in the 2-3, 3-4 or 4-5 position to a phenyl, (C₄-C₈)cycloalkyl or heterocyclic group, which may be optionally substituted, W represents X—Y or Y—X, wherein:
X represents 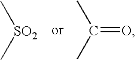
and Y represents oxygen or N—R₃, n represents zero or an integer from 1 to 6,
G, R₁, R₂ and R₃ are as defined in the description, its enantiomers and diastereoisomers, and addition salts thereof with a pharmaceutically acceptable acid or base, and medicinal products containing the same which are useful for the treatment of cancerous diseases.

The present invention relates to new tricyclic azepine compounds, to a process for their preparation, to pharmaceutical compositions containing them and also to the use thereof as anti-cancer agents.

Anti-cancer therapeutic requirements call for the constant development of new anti-tumour agents with the aim of obtaining medicaments that are simultaneously more active and better tolerated.

Besides the fact that the compounds of the invention are new, they have valuable anti-tumour properties.

Compounds having a closely related structure have been described in the literature, especially amino-dihydro-dibenzothiazepine compounds in the field of psychoneurotic disorders (patent specification FR 2 104 728), dihydro-pyridobenzothiadiazepine compounds as psychotropic agents (patent specification U.S. Pat. No. 3,274,058) and antivirals (patent specification WO 94 17075), dihydro-pyridobenzodiazepine and dihydro-dipyridodiazepine compounds as antivirals (patent specifications EP 0 393 530, U.S. Pat. No. 5,620,974 and EP 0 393 604), and amino-dihydro-dibenzoazepine compounds as anti-convulsive agents (Eur. J. Med. Chem. 1988, 23 (5), 473-6; J. Pharm. Pharmacol. 1969, 21 (8), 520-530). Finally, other aryl-pyrido-diazepine and thiodiazepine compounds of closely related structure have been described as selective inhibitors of HIV (Antiviral Research 1996, 30 (2,3), 109-124; Bioorg. Med. Chem. Lett. 1995, 5 (14), 1461-6; J. Med. Chem. 1991, 34 (7), 2231-41; and Farmaco, Ed. Scientifica 1985, 40 (6), 391-403).

However, no cytotoxic activity has ever been described for those compounds.

More specifically, the present invention relates to compounds of formula (I): 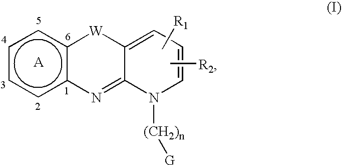
wherein: 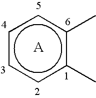
represents a benzo or pyrido group optionally fused in the 2-3, 3-4 or 4-5 position, it being understood that the nitrogen atom of the pyrido group occupies any of positions 2 to 5 in the ring, which ring is optionally substituted by one or more identical or different atoms or groups selected from halogen atoms and the groups hydroxy, linear or branched (C₁-C₆)alkyl, linear or branched (C₁-C₆)alkoxy, linear or branched (C₁-C₆)trihaloalkyl, amino (optionally substituted at the nitrogen atom by one or two linear or branched (C₁-C₆)alkyl groups), nitro, linear or branched (C₁-C₆)acyl and (C₁-C₂)alkylenedioxy,
W represents a group X—Y or Y—X wherein:
   X represents a group 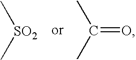
   and Y represents an oxygen atom or a group N—R₃ wherein R₃ represents a hydrogen atom, a linear or branched (C₁-C₆)alkyl group, an aryl-(C₁-C₆)alkyl group wherein the alkyl group is linear or branched, -Alk-Z-R or -Alk-Z-Alk′-Z′-R wherein Alk and Alk′ represent, each independently of the other, a linear or branched (C₁-C₆)alkylene group or a linear or branched (C₂-C₆)alkenylene group, Z and Z′ represent, each independently of the other, an oxygen or sulphur atom or a group —N(R′)—, and R and R′, which are the same or different, represent a linear or branched (C₁-C₆)alkyl group,
n represents zero or an integer wherein 1≦n≦6,
G represents a hydrogen atom, an aryl group or a heteroaryl group,
R₁ and R₂, which are the same or different, represent a hydrogen or halogen atom or a hydroxy group, a linear or branched (C₁-C₆)alkyl group, a linear or branched (C₁-C₆)alkoxy group, a linear or branched (C₁-C₆)trihaloalkyl group, an amino group (optionally substituted at the nitrogen atom by one or two linear or branched (C₁-C₆)alkyl groups), a nitro group, a linear or branched (C₁-C₆)acyl group or a (C₁-C₂)alkylenedioxy group,
   to their enantiomers and diastereoisomers, and also to addition salts thereof with a pharmaceutically acceptable acid or base,
   with the proviso that:
n is other than zero when g represents a hydrogen atom,
when G represents a hydrogen atom and Y represents a group N—R₃, then R₃ represents a hydrogen atom, a linear or branched (C₂-C₆)alkyl group or an aryl-(C₁-C₆)alkyl group wherein the alkyl group is linear or branched,
when G represents a hydrogen atom and W represents one of the two NR₃C(O) groups wherein R₃ represents an ethyl or benzyl group, n is other than 1, 2 or 3,
the compounds of formula (I) are other than 1-benzyl-5,10-dimethyl-1,5-dihydro-6H-pyrido[2,3-b][1,4]benzodiazepin-6-one, ethyl 1,2-dimethyl-5-oxo-5,6-dihydro-1H-pyrido[2,3-b][1,5]benzodiazepine 3-carboxylate, 3-acetyl-1′-ethyl-2-methyl-1,6-dihydro-5H-pyrido[2,3-b][1,5]benzodiazepin-5-one, 2-amino-1′-methyl-5-oxo-5,6-dihydro-1H-pyrido [2,3-b][1,5]benzodiazepine-3-carbonitrile and ethyl 2-amino-1′-methyl-5-oxo-5,6-dihydro-1H-pyrido[2,3-b][1,5]benzodiazepine 3-carboxylate,
   it being understood that:
an aryl group means phenyl, biphenyl, naphthyl, tetrahydronaphthyl, each of those groups being optionally substituted by one, two or three identical or different atoms or groups selected from halogen atoms and the groups linear or branched (C₁-C₆)alkyl, hydroxy, linear or branched (C₁-C₆)alkoxy, linear or branched (C₁-C₆)trihaloalkyl and amino (optionally substituted at the nitrogen atom by one or two linear or branched (C₁-C₆)alkyl groups), nitro, linear or branched (C₁-C₆)acyl, linear or branched (C₁-C₆)alkylcarbonylamino, (C₁-C₂)alkylenedioxy, phenyloxy, benzyloxy, linear or branched amino-(C₁-C₆)alkoxy, linear or branched (C₁-C₆)alkylamino-(C₁-C₆)alkoxy and linear or branched di(C₁-C₆)alkylamino-(C₁-C₆)alkoxy,
a heteroaryl group means a mono- or bi-cyclic, aromatic, 5- to 12-membered group containing one, two or three hetero atoms selected from oxygen, nitrogen and sulphur, it being understood that the heteroaryl group may be optionally substituted by one or more identical or different atoms or groups selected from halogen atoms and the groups linear or branched (C₁-C₆)alkyl, hydroxy, linear or branched (C₁-C₆)alkoxy, linear or branched (C₁-C₆)trihaloalkyl and amino (optionally substituted by one or more linear or branched (C₁-C₆)alkyl groups), nitro, linear or branched (C₁-C₆)acyl, linear or branched (C₁-C₆)alkylcarbonylamino, (C₁-C₂)alkylenedioxy, phenyloxy, benzyloxy, linear or branched amino-(C₁-C₆)alkoxy, linear or branched (C₁-C₆)alkylamino-(C₁-C₆)alkoxy and linear or branched di(C₁-C₆)alkylamino-(C₁-C₆)alkoxy,
a group 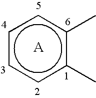
optionally fused in the 2-3, 3-4 or 4-5 position means that the benzo or pyrido group is optionally fused to a phenyl, (C₄-C₈)cycloalkyl or heterocyclic group in the position 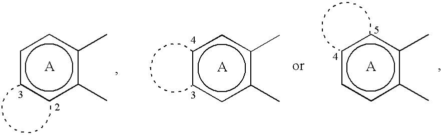
provided that, when 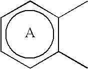
represents a pyrido group, the nitrogen atom is not a point of attachment to the fused ring,
an alkylene group means a bivalent radical of a saturated hydrocarbon chain,
an alkenylene group means a bivalent radical of a hydrocarbon chain containing from 1 to 3 double bonds,
a (C₄-C₈)cycloalkyl group means a cyclobutane, cyclopentane, cyclohexane, cycloheptane or cyclooctane group, and
a heterocyclic group means a saturated or unsaturated, 5- to 7-membered, monocyclic group containing from one to three hetero atoms selected from nitrogen, oxygen and sulphur.

Among the heteroaryl groups there may be mentioned, without implying any limitation, the groups thienyl, pyridyl, furyl, pyrrolyl, imidazolyl, oxazolyl, isoxazolyl, thiazolyl, isothiazolyl, quinolyl, isoquinolyl and pyrimidinyl.

Among the heterocyclic groups there may be mentioned, without implying any limitation, the groups thienyl, pyridyl, pyranyl, furyl, pyrrolyl, imidazolyl, thiazolyl, pyrimidyl, piperidyl, piperazinyl and morpholino.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid and camphoric acid.

Among the pharmaceutically acceptable bases there may be mentioned, without implying any limitation, sodium hydroxide, potassium hydroxide, triethylamine and tert-butylamine.

The term “aryl” relating to the group G as defined in formula (I) is preferably a substituted phenyl group.

An advantageous embodiment of the invention relates to compounds of formula (I) wherein G represents an aryl or heteroaryl group, more advantageously an aryl group.

An especially advantageous aspect relates to compounds of formula (I) wherein G represents a phenyl group substituted by one, two or three groups selected from linear or branched (C₁-C₆)alkoxy, benzyloxy and hydroxy. More advantageously, the groups substituting the phenyl group G are linear or branched (C₁-C₆)alkoxy or hydroxy.

Preferred compounds of formula (I) are those wherein X represents 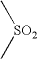
and Y represents N—R₃ or O.

Other preferred compounds of the invention relate to compounds of formula (I) wherein X represents 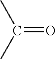
and Y represents N—R₃ or O.

In compounds of formula (I) wherein Y represents N—R₃, R₃ preferably represents a linear or branched (C₁-C₆)alkyl group, more especially a methyl group.

Other preferred compounds are those wherein R₃ represents a hydrogen atom or aryl-(C₁-C₆)alkyl wherein the alkyl group is linear or branched, more especially a hydrogen atom.

Advantageously, the invention relates to compounds of formula (I) wherein 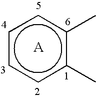
represents a group 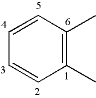
optionally substituted by 1, 2 or 3 identical or different atoms or groups selected from halogen atoms and the groups hydroxy, linear or branched (C₁-C₆)alkyl, linear or branched (C₁-C₆)alkoxy, linear or branched (C₁-C₆)trihaloalkyl, amino (optionally substituted at the nitrogen atom by one or two linear or branched (C₁-C₆)alkyl groups), nitro, linear or branched (C₁-C₆)acyl and (C₁-C₂)alkylenedioxy.

Preferably, the substituents are located in the 3- or 4-position of the group 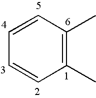
and are selected from halogen atoms and the groups linear or branched (C₁-C₆)alkyl, more especially methyl, linear or branched (C₁-C₆)alkoxy, more especially methoxy, and linear or branched (C₁-C₆)trihaloalkyl, more especially trifluoromethyl.

Another advantageous aspect relates to compounds of formula (I) wherein R₁ and R₂, which are the same or different, represent a hydrogen or halogen atom or a linear or branched (C₁-C₆)alkyl group, a linear or branched (C₁-C₆)alkoxy group or a linear or branched (C₁-C₆)trihaloalkyl group.

Among the preferred compounds there may be mentioned:
1-[2-(4-methoxyphenyl)ethyl]-6-methyl-1,6-dihydropyrido[3,2-c][2,1,5]benzothiadiazepine 5,5-dioxide,
1-[2-(4-methoxyphenyl)ethyl]-5-methyl-1,5-dihydropyrido[3,2-c][1,2,5]benzothiadiazepine 6,6-dioxide,
1-[2-(4-methoxyphenyl)ethyl]-1H-pyrido[3,2-c][1,2,5]benzoxathiazepine 5,5-dioxide,
6-[(2-methoxyethoxy)methyl]-1-[2-(4-methoxyphenyl)ethyl]-1,6-dihydropyrido[3,2-c][2,1,5]benzothiadiazepine 5,5-dioxide,
1-[2-(4-methoxyphenyl)ethyl]-1,6-dihydropyrido[3,2-c][2,1,5]benzothiadiazepine 5,5-dioxide,
4-[2-(5,5-dioxido-1H-pyrido[3,2-c][1,2,5]benzoxathiazepin-1-yl)ethyl]phenol,
1-[2-(2-methoxyphenyl)ethyl]-1H-pyrido[3,2-c][1,2,5]benzoxathiazepine 5,5-dioxide,
1-{2-[3-(benzyloxy)-4-methoxyphenyl]ethyl}-1H-pyrido[3,2-c][1,2,5]benzoxathiazepine 5,5-dioxide,
5-[2-(5,5-dioxido-1H-pyrido[3,2-c][1,2,5]benzoxathiazepin-1-yl)ethyl]-2-methoxyphenol.

The invention relates also to a process for the preparation of compounds of formula (I), which process is characterised in that in a basic medium:
a compound of formula (II): 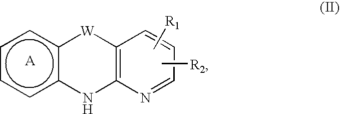
wherein W, A, R₁ and R₂ are as defined for formula (I),
   is reacted with a compound of formula (III):
      Z₂-(CH₂)ₙ-G (III),
   wherein n and G are as defined for formula (I) and Z₂ represents a nucleofugal group,
      to yield the compound of formula (I), which is purified, where necessary, according to a conventional purification technique, which is separated, if desired, into its stereoisomers according to a conventional separation technique, and which is converted, if desired, into their addition salts with a pharmaceutically acceptable acid or base.

The compound of formula (II) is obtained:
either starting from condensation of the reagent (IV): 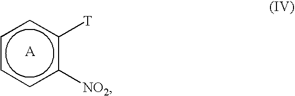
wherein A is as defined for formula (I) and T represents a group X—Cl or Y₁—H, wherein X is as defined for formula (I) and Y₁ represents an oxygen atom or a group N—R₄ wherein R₄ represents a hydrogen atom, a linear or branched (C₁-C₆)alkyl group or a protecting group for the amino function,
with a compound of formula (V): 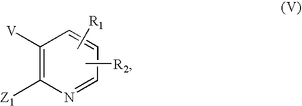
wherein R₁ and R₂ are as defined for formula (I), Z₁ represents a halogen atom and V either represents a group Y₁—H when T represents a group X—Cl or represents a group X—Cl when T represents a group Y₁—H,
to yield the compound of formula (VIa): 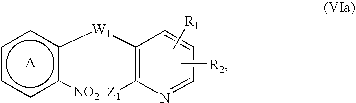
wherein A, R₁, R₂ and Z₁ are as defined hereinbefore and W₁ represents a group X—Y₁ or Y₁—X wherein X and Y₁ are as defined hereinbefore,
   which compound of formula (VIa), when Y₁ represents a group NH, may be coupled, in a basic medium, with a halogenated compound R₃Hal wherein R₃ is as defined for formula (I) to yield the compound of formula (VIb) or (VIc): 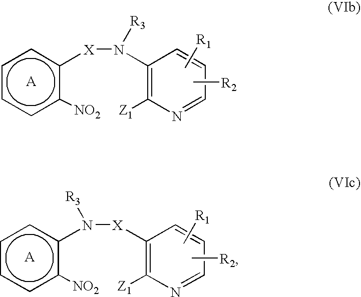
   wherein A, R₁, R₂, R₃, X and Z₁ are as defined hereinbefore,
   it being possible to represent the compounds of formulae (VIa), (VIb) and (VIc) by the general formula (VI): 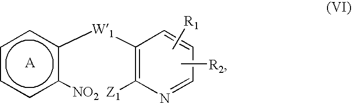
   wherein A, R₁, and R₂ are as defined for formula (I), Z, is as defined hereinbefore and W′₁ represents a group X—Y′₁ or Y′₁—X wherein X is as defined for formula (I) and Y′₁ represents an oxygen atom or a group N—R′₄ wherein R′₄ represents a protecting group for the amino function,
the NO₂ function of which compound of formula (VI) is then converted by conventional reactions of organic chemistry to yield the compound of formula (VII): 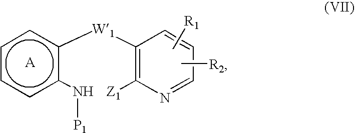
wherein A, W′₁, R₁, R₂ and Z₁ are as defined hereinbefore and P₁ represents a hydrogen atom or a protecting group for the amino function,
which is then converted by a cyclisation reaction in an acid or basic medium, optionally followed by one or two deprotection reactions and then optionally by an alkylation reaction, into the compound of formula (II),
   or starting from condensation of the compound (VIII): 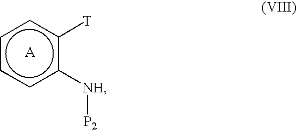
   wherein A and T are as defined hereinbefore and P₂ represents a hydrogen atom or a protecting group for the amino function,
with the compound of formula (V) described hereinbefore to yield the compound of formula (IX): 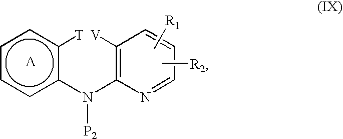
wherein A, T, V, P₂, R₁ and R₂ are as defined hereinbefore,
which is then converted by a cyclisation reaction in an acid or basic medium, optionally followed by one or two deprotection reactions and then optionally by an alkylation reaction, into the compound of formula (II).

Besides the fact that the compounds of the present invention are new, they have valuable pharmacological properties. They have cytotoxic properties which make them useful in the treatment of cancers.

The invention relates also to pharmaceutical compositions comprising at least one compound of formula (I) as active ingredient together with one or more appropriate, inert, non-toxic excipients. Among the pharmaceutical compositions according to the invention, there may be mentioned, more especially, those that are suitable for oral, parenteral (intravenous, intramuscular or subcutaneous) or nasal administration, tablets or dragées, sublingual tablets, gelatin capsules, lozenges, suppositories, creams, ointments, dermal gels, injectable preparations, drinkable suspensions etc.

The dosage used may be varied according to the nature and severity of the condition, the administration route and the age and weight of the patient and any associated treatments and varies from 1 to 500 mg per day in one or more administrations.

The Examples that follow illustrate the invention and do not limit it in any way.

The starting compounds used are known compounds or are prepared according to known methods of preparation.

The structures of the compounds described in the Examples were determined according to customary spectrometric and spectroscopic techniques.

### Preparation A: 6-Methyl-6,11-dihydropyrido[3,2-c][2,1,5]benzothiadiazepine 5,5-dioxide

### Step A: 2-Chloro-N-(2-nitrophenyl)-3-pyridinesulphonamide

The product is obtained according to the procedure described in the publication J. Med. Chem., 1991, 34 (4), 1356-1362, starting from 2-chloro-3-pyridinesulphochloride and 2-nitroaniline.

### Step B: 6-Methyl-6,11-dihydropyrido[3,2-c][2,1,5]benzothiadiazepine 5,5-dioxide

2-Chloro-N-methyl-N-(2-nitrophenyl)-3-pyridinesulphonamide is synthesised by N-alkylation of the compound prepared in the previous Step, using methyl iodide in a basic medium (procedure described in the publication J. Med. Chem., 1991, 34 (4), 1356-1362). 2-Chloro-N-methyl-N-(2-nitrophenyl)-3-pyridinesulphonamide (0.005 mol) is then dissolved in concentrated acetic acid (20 ml), and iron (0.025 mol) is added. Evaporate under reduced pressure, take up in water and extract with ethyl acetate. Dry over sodium sulphate and then evaporate under reduced pressure. Recrystallise the resulting precipitate from ethanol.

Melting point 180° C.

### Preparation B: 5-Methyl-5,11-dihydropyrido[3,2-c][1,2,5]benzothiadiazepine 6,6-dioxide

### Step A: N-(2-Chloro-3-pyridyl)-2-nitrobenzenesulphonamide

Add, in fractions, to a solution of 2-nitrobenzenesulphochloride (0.001 mol) in pyridine (3 ml), 3-amino-2-chloropyridine (0.001 mol). Heat at 70° C. for 2 hours. After cooling, take up the solution in water. Extract with ethyl acetate and then wash the organic phase with 1N hydrochloric acid. Dry over sodium sulphate, filter and evaporate the organic phases under reduced pressure. The sulphonamide is then recrystallised from ethanol.

Melting point: 145-147° C.

### Step B: N-(2-{[(2-Chloro-3-pyridyl)(methyl)amino]sulphonyl}phenyl)acetamide

N-(2-Chloro-3-pyridyl)-N-methyl-2-nitrobenzenesulphonamide is synthesised by N-alkylation of the compound prepared in the previous Step, using methyl iodide in a basic medium (procedure described in the publication J. Med. Chem., 1991, 34 (4), 1356-1362). N-(2-Chloro-3-pyridyl)-N-methyl-2-nitrobenzenesulphonamide (0.001 mol) is then hydrogenated over Raney nickel (0.003 mol) in absolute ethanol (150 ml) at atmospheric pressure and ambient temperature. The nickel is removed, the solvent is evaporated off under reduced pressure and then acetic anhydride (20 ml) is added to the crude product. The solution is stirred for 12 hours. The mixture is then diluted with water, extracted with dichloromethane, dried and recrystallised.

Melting point 116-118° C.

### Step C: 5-Methyl-5,11-dihydropyrido[3,2-c][1,2,5]benzothiadiazepine 6,6-dioxide

Reflux a solution of the compound prepared in the previous Step (0.04 mol), potassium carbonate (0.008 mol) and copper (0.10 g) in dimethylformamide (20 ml) for 8 hours. Filter and evaporate under reduced pressure. Take up in water, extract the solution with dichloromethane, dry the organic phases over sodium sulphate, evaporate under reduced pressure and recrystallise from ethanol.

Melting point 203-204° C.

### Preparation C: 11H-Pyrido[3,2-c][1,2,5]benzoxathiazepine 5,5-dioxide

### Step A: 2-Aminophenyl 2-chloro-3-pyridinesulphonate

Add a solution of 2-chloro-3-pyridinesulphochloride (0.019 mol) in dichloromethane (30 ml) dropwise to a mixture of 2-aminophenol (0.019 mol) and triethylamine (0.022 mol). Stir at ambient temperature for 24 hours. Wash the solution with 1N hydrochloric acid and then with water. Dry, filter and evaporate the organic phases under reduced pressure. 2-Aminophenyl 2-chloro-3-pyridinesulphonate is used as such in the following cyclisation step.

### Step B: 11H-Pyrido[3,2-c][1,2,5]benzoxathiazepine 5,5-dioxide

11H-Pyrido[3,2-c][1,2,5]benzoxathiazepine 5,5-dioxide is obtained by refluxing, in absolute ethanol, the compound prepared in the previous Step. Then evaporate off the solvents, take up in dichloromethane and wash with 7% ammonium hydroxide solution and then with water. Dry over sodium sulphate. Evaporate under reduced pressure and recrystallise from ethanol.

Melting point 208-209° C. (ethanol)

### Preparation D: Pyrido[3,2-c][1,5]benzoxazepin-5(11H)-one

### Step A: 2-(2-Hydroxyanilino)nicotinic acid

Reflux a mixture of 2-chloronicotinic acid (0.032 mol) and 2-aminophenol (0.038 mol) in xylene (25 ml) for three hours. After reaction, filter off, under suction, the precipitate formed. The black precipitate obtained is recrystallised from water in the presence of carbon black.

Melting point 225-227° C. degradation (H₂O)

### Step B: Pyrido[3,2-c][1,5]benzoxazepin-5(11H)-one

Bring a solution of 2-(2-hydroxyaniline)nicotinic acid (0.009 mol) in 250 ml of dichloromethane to 0° C. Add, dropwise, 1-[3-(dimethylamino)propyl]-3-ethylcarbodiimide hydrochloride (0.010 mol) dissolved in 50 ml of dichloromethane. Stir for one hour at 0° C., then return to ambient temperature and stir overnight. Filter, wash with water, evaporate under reduced pressure and recrystallise from propanol.

Melting point 189-191° C. (propanol)

### Preparation E: 6-[(2-Methoxyethoxy)methyl]-6,11-dihydropyrido[3,2-c][2,1,5]benzothiadiazepine 5,5-dioxide

The expected compound is obtained according to the procedure described in Preparation A, replacing the methyl iodide in Step B by methoxyethoxymethyl chloride.

Melting point 119-121° C.

### Preparation F: 6-Methyl-6,11-dihydro-5H-pyrido[2,3-b][1,5]benzodiazepin-5-one

The expected compound is obtained according to the procedure described in Preparation D, replacing the 2-aminophenol in Step A by 2-aminoaniline. The intermediate product, 6,11-dihydro-5H-pyrido[2,3-b][1,5]benzodiazepin-5-one, is N-alkylated using methyl iodide in a basic medium (procedure described in the publication J. Med. Chem., 1991, 34 (4), 1356-1362).

### EXAMPLE 1

### 1-(4-Methoxybenzyl)-6-methyl-1,6-dihydropyrido[3,2-c][2,1,5]benzothiadiazepine 5,5-dioxide

To a suspension of sodium hydride (60%) (0.012 mol) in dimethylformamide (20 ml) add, dropwise, a solution of the azepine prepared in Preparation A (0.004 mol) in dimethylformamide. Stir at 60° C. for 2 hours. Add a solution of 4-methoxybenzyl chloride (0.012 mol) dropwise. Stir overnight at 60° C. Evaporate the solution to dryness, take up the residue in water and extract with dichloromethane. Dry, filter and evaporate the organic phases under reduced pressure. Purify the resulting oil by preparative HPLC (column of 50 mm diameter filled with 250 g of normal Lichoprep Si 60 MERCK silica (15/25 μm)) and recrystallise from ethanol.

Melting point 127-129° C. (ethanol)

### EXAMPLE 2

### 1-[2-(4-Methoxyphenyl)ethyl]-6-methyl-1,6-dihydropyrido[3,2-c][2,1,5]benzothiadiazepine 5,5-dioxide

The expected compound is obtained according to the procedure described in Example 1, replacing the 4-methoxybenzyl chloride by 4-methoxyphenylethyl methanesulphonate.

Melting point 105-107° C. (ethanol)

### EXAMPLE 3

### 1-[3-(4-Methoxyphenyl)propyl]-6-methyl-1,6-dihydropyrido[3,2-c][2,1,5]benzothiadiazepine 5,5-dioxide

The expected compound is obtained according to the procedure described in Example 1, replacing the 4-methoxybenzyl chloride by 3-(4-methoxyphenyl)propyl methanesulphonate.

Melting point 50-55° C. (isopropanol)

### EXAMPLE 4

### 9-Chloro-1-(4-methoxybenzyl)-6-methyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 4-chloro-2-nitroaniline.

Melting point 149° C. (ethanol)

### EXAMPLE 5

### 9-Chloro-1-[2-(4-methoxyphenyl)ethyl]-6-methyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 4-chloro-2-nitroaniline, and the 4-methoxybenzyl chloride is replaced by 4-methoxyphenylethyl methanesulphonate.

Melting point 163° C. (ethanol)

### EXAMPLE 6

### 8-Chloro-1-(4-methoxybenzyl)-6-methyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 5-chloro-2-nitroaniline.

Melting point 109° C. (ethanol)

### EXAMPLE 7

### 8-Chloro-1-[2-(4-methoxyphenyl)ethyl]-6-methyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 5-chloro-2-nitroaniline, and the 4-methoxybenzyl chloride is replaced by 4-methoxyphenylethyl methanesulphonate.

Melting point 100-101° C. (ethanol)

### EXAMPLE 8

### 1-(4-Methoxybenzyl)-6,9-dimethyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 4-methyl-2-nitroaniline.

Melting point 90-92° C. (ethanol)

### EXAMPLE 9

### 1-[2-(4-Methoxyphenyl)ethyl]-6,9-dimethyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 4-methyl-2-nitroaniline, and the 4-methoxybenzyl chloride is replaced by 4-methoxyphenylethyl methanesulphonate.

Melting point 156-157° C. (ethanol)

### EXAMPLE 10

### 9-Methoxy-1-(4-methoxybenzyl)-6-methyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 4-methoxy-2-nitroaniline.

Melting point 95-96° C. (ethanol)

### EXAMPLE 11

### 9-Methoxy-1-[2-(4-methoxyphenyl)ethyl]-6-methyl-1,6-dihydropyrido[3,2-c][2,1,5]benzothiadiazepine 5,5-dioxide

The expected compound is obtained according to the procedure described in Example 1, replacing the 2-nitroaniline in Preparation A, Step A, by 4-methoxy-2-nitroaniline, and the 4-methoxybenzyl chloride is replaced by 4-methoxyphenylethyl methanesulphonate.

Melting point 146° C. (ethanol)

### EXAMPLE 12

### 1-[2-(4-Methoxyphenyl)ethyl]-6-[2-(N,N-diethylamino)ethyl]-1,6-dihydropyrido[3,2-c][2,1,5]benzothiadiazepine 5,5-dioxide

The expected compound is obtained according to the procedure described in Example 1, replacing the methyl iodide used in the N-alkylation in Preparation A, Step B, by 1-chloro-2-(N,N-diethylamino)ethane hydrochloride, and the 4-methoxybenzyl chloride is replaced by 4-methoxyphenylethyl methanesulphonate.

Melting point 92° C. decomposition (ethanol)

### EXAMPLE 13

### 1,6-Bis(4-methoxybenzyl)-1,6-dihydropyrido[3,2-c][2,1,5]benzothiadiazepine 5,5-dioxide

The expected compound is obtained according to the procedure described in Example 1, replacing the methyl iodide used in the N-substitution in Preparation A, Step B, by 4-methoxybenzyl chloride.

Melting point 95-98° C. (ethanol)

### EXAMPLE 14

### 6-(4-Methoxybenzyl)-1-[2-(4-methoxyphenyl)ethyl]-1,6-dihydropyrido[3,2-c][2,1,5]benzothiadiazepine 5,5-dioxide

The expected compound is obtained according to the procedure described in Example 1, replacing the methyl iodide used in the N-substitution in Preparation A, Step B, by 4-methoxybenzyl chloride, and the 4-methoxybenzyl chloride is replaced by 4-methoxyphenylethyl methanesulphonate.

Melting point 67° C. (ethanol)

### EXAMPLE 15

### 1-(4-Methoxybenzyl)-5-methyl-1,5-dihydropyrido[3,2-c][1,2,5]benzothiadiazepine 6,6-dioxide

The expected compound is obtained according to the procedure described in Example 1, starting from the compound prepared in Preparation B, Step C.

Melting point 174-177° C. (ethanol)

### EXAMPLE 16

### 1-[2-(4-Methoxyphenyl)ethyl]-5-methyl-1,5-dihydropyrido[3,2-c][1,2,5]benzothiadiazepine 6,6-dioxide

The expected compound is obtained according to the procedure described in Example 15, except that the 4-methoxybenzyl chloride is replaced by 4-methoxyphenylethyl methanesulphonate.

Melting point 181-183° C. (ethanol)

### EXAMPLE 17

### 1-[3-(4-Methoxyphenyl)propyl]-5-methyl-1,5-dihydropyrido[3,2-c][1,2,5]benzothiadiazepine 6,6-dioxide

The expected compound is obtained according to the procedure described in Example 15, except that the 4-methoxybenzyl chloride is replaced by 3-(4-methoxyphenyl)propyl methanesulphonate.

Melting point 94-96° C. (ethanol)

### EXAMPLE 18

### 5-Methyl-1-(3,4,5-trimethoxybenzyl)-1,5-dihydropyrido[3,2-c][1,2,5]benzothiadiazepine 6,6-dioxide

The expected compound is obtained according to the procedure described in Example 15, except that the 4-methoxybenzyl chloride is replaced by 3,4,5-trimethoxybenzyl methanesulphonate.

Melting point 178-180° C. (ethanol)

### EXAMPLE 19

### 9-Chloro-1-(4-methoxybenzyl)-5-methyl-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride.

Mass spectrum: [M⁺]=415

### EXAMPLE 20

### 9-Chloro-1-[2-(4-methoxyphenyl)ethyl]-5-methyl-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride, and the 4-methoxybenzyl chloride is replaced by 4-methoxyphenylethyl methanesulphonate.

Mass spectrum: [M⁺]=429

### EXAMPLE 21

### 9-Chloro-1-[3-(4-methoxyphenyl)propyl]-5-methyl-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride, and the 4-methoxybenzyl chloride is replaced by 4-methoxyphenylpropyl methanesulphonate.

Melting point 112-113° C. (ethanol)

### EXAMPLE 22

### 9-Chloro-5-methyl-1-[2-(3,4,5-trimethoxyphenyl)ethyl]-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride, and the 4-methoxybenzyl chloride is replaced by 2-(3,4,5-trimethoxyphenyl)ethyl methanesulphonate.

Melting point 203-204° C. (ethanol)

### EXAMPLE 23

### 1-[4-(Benzyloxy)benzyl]-9-chloro-5-methyl-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride, and the 4-methoxybenzyl chloride is replaced by 4-benzyloxybenzyl chloride.

Melting point 86° C. (ethanol)

### EXAMPLE 24

### 1-{2-[4-(Benzyloxy)phenyl]ethyl}-9-chloro-5-methyl-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride, and the 4-methoxybenzyl methanesulphonate is replaced by 2-(4-benzyloxyphenyl)ethyl chloride.

Melting point 121-122° C. (ethanol)

### EXAMPLE 25

### 9-Chloro-1,5-bis(4-methoxybenzyl)-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride, and the methyl iodide used in the N-substitution of the compound is replaced by 4-methoxybenzyl chloride.

Melting point 70-71° C. (ethanol)

### EXAMPLE 26

### 9-Chloro-5-(4-methoxybenzyl)-1-[2-(4-methoxyphenyl)ethyl]-1,5-dihydropyrido[3,2-c][1,2,5]benzothiadiazepine 6,6-dioxide

The expected compound is obtained according to the procedure described in Example 15, replacing the 2-nitrobenzenesulphochloride in Preparation B, Step A, by 4-chloro-2-nitrobenzenesulphochloride; the methyl iodide used in the N-substitution of the compound is replaced by 4-methoxybenzyl methanesulphonate, and the 4-methoxybenzyl chloride is replaced by 2-(4-benzyloxyphenyl)ethyl chloride.

Melting point 168-169° C. (ethanol)

### EXAMPLE 27

### 8-Chloro-1-(4-methoxybenzyl)-5-methyl-1,5-dihydropyrido[3,2-c][1,2,5]benzothiadiazepine 6,6-dioxide

The expected compound is obtained according to the procedure described in Example 19, starting from 5-chloro-2-nitrobenzenesulphochloride instead of from 4-chloro-2-nitrobenzenesulphochloride in Preparation B.

Melting point 162-163° C. (ethanol)

### EXAMPLE 28

### 8-Chloro-1-[2-(4-methoxyphenyl)ethyl]-5-methyl-1,5-dihydropyrido[3,2-c][1,2,5]benzothiadiazepine 6,6-dioxide

The expected compound is obtained according to the procedure described in Example 19, starting from 5-chloro-2-nitrobenzenesulphochloride instead of from 4-chloro-2-nitrobenzenesulphochloride in Preparation B, and from 2-(4-methoxyphenyl)ethyl methanesulphonate instead of from 4-methoxybenzyl chloride.

Melting point 186-188° C. (ethanol)

### EXAMPLE 29

### 8-Chloro-1-[3-(4-methoxyphenyl)propyl]-5-methyl-1,5-dihydropyrido[3,2-c][1,2,5]benzothiadiazepine 6,6-dioxide

The expected compound is obtained according to the procedure described in Example 21, starting from 5-chloro-2-nitrobenzenesulphochloride instead of from 4-chloro-2-nitrobenzenesulphochloride in Preparation B.

Melting point 62-65° C. (isopropanol)

### EXAMPLE 30

### 1-(4-Methoxybenzyl)-5,9-dimethyl-1,5-dihydropyrido[3,2-c][1,2,5]benzothiadiazepine 6,6-dioxide

The expected compound is obtained according to the procedure described in Example 19, starting from 4-methyl-2-nitrobenzenesulphochloride instead of from 4-chloro-2-nitrobenzenesulphochloride in Preparation B.

Melting point 135-136° C. (ethanol)

### EXAMPLE 31

### 1-[2-(4-Methoxyphenyl)ethyl]-5,9-dimethyl-1,5-dihydropyrido[3,2-c][1,2,5]benzothiadiazepine 6,6-dioxide

The expected compound is obtained according to the procedure described in Example 20, starting from 4-methyl-2-nitrobenzenesulphochloride instead of from 4-chloro-2-nitrobenzenesulphochloride in Preparation B.

Melting point 128° C. (ethanol)

### EXAMPLE 32

### 1-[3-(4-Methoxyphenyl)propyl]-5,9-dimethyl-1,5-dihydropyrido[3,2-c][1,2,5]benzothiadiazepine 6,6-dioxide

The expected compound is obtained according to the procedure described in Example 21, starting from 4-methyl-2-nitrobenzenesulphochloride instead of from 4-chloro-2-nitrobenzenesulphochloride in Preparation B.

Melting point 130-131° C. (ethanol)

### EXAMPLE 33

### 9-Methoxy-1-(4-methoxybenzyl)-5-methyl-1,5-dihydropyrido[3,2-c][1,2,5]benzothiadiazepine 6,6-dioxide

The expected compound is obtained according to the procedure described in Example 19, starting from 4-methoxy-2-nitrobenzenesulphochloride instead of from 4-chloro-2-nitrobenzenesulphochloride in Preparation B.

Melting point 179-180° C. (ethanol)

### EXAMPLE 34

### 9-Methoxy-1-[2-(4-methoxyphenyl)ethyl]-5-methyl-1,5-dihydropyrido[3,2-c][1,2,5]benzothiadiazepine 6,6-dioxide

The expected compound is obtained according to the procedure described in Example 20, starting from 4-methoxy-2-nitrobenzenesulphochloride instead of from 4-chloro-2-nitrobenzenesulphochloride in Preparation B.

Melting point 65-68° C. (ethanol)

### EXAMPLE 35

### 9-Methoxy-1-[2-(4-methoxyphenyl)propyl]-5-methyl-1,5-dihydropyrido[3,2-c][1,2,5]benzothiadiazepine 6,6-dioxide

The expected compound is obtained according to the procedure described in Example 21, starting from 4-methoxy-2-nitrobenzenesulphochloride instead of from 4-chloro-2-nitrobenzenesulphochloride in Preparation B.

Melting point 128-131° C. (isopropanol)

### EXAMPLE 36

### 1-(4-Methoxybenzyl)-5-methyl-9-(trifluoromethyl)-1,5-dihydropyrido[3,2-c][1,2,5]benzothiadiazepine 6,6-dioxide

The expected compound is obtained according to the procedure described in Example 19, starting from 4-trifluoromethyl-2-nitrobenzenesulphochloride instead of from 4-chloro-2-nitrobenzenesulphochloride in Preparation B.

Melting point 142-143° C. (ethanol)

### EXAMPLE 37

### 1-[2-(4-Methoxyphenyl)ethyl]-5-methyl-9-(trifluoromethyl)-1,5-dihydropyrido[3,2-c][1,2,5]benzothiadiazepine 6,6-dioxide

The expected compound is obtained according to the procedure described in Example 20, starting from 4-trifluoromethyl-2-nitrobenzenesulphochloride instead of from 4-chloro-2-nitrobenzenesulphochloride in Preparation B.

Melting point 43-44° C. (methanol)

### EXAMPLE 38

### 1-[3-(4-Methoxyphenyl)propyl]-5-methyl-9-(trifluoromethyl)-1,5-dihydropyrido[3,2-c][1,2,5]benzothiadiazepine 6,6-dioxide

The expected compound is obtained according to the procedure described in Example 21, starting from 4-trifluoromethyl-2-nitrobenzenesulphochloride instead of from 4-chloro-2-nitrobenzenesulphochloride in Preparation B.

Melting point 144-145° C. (ethanol)

### EXAMPLE 39

### 1-{2-[4-(Benzyloxy)phenyl]ethyl}-5-methyl-1,5-dihydropyrido[3,2-c][1,2,5]benzothiadiazepine 6,6-dioxide

The expected compound is obtained according to the procedure described in Example 24, starting from 2-nitrobenzenesulphochloride instead of from 4-chloro-2-nitrobenzenesulphochloride in Preparation B.

Melting point 133-134° C. (ethanol)

### EXAMPLE 40

### 1-[2-(4-Phenol)ethyl]-5-methyl-1,5-dihydropyrido[3,2-c][1,2,5]benzothiadiazepine 6,6-dioxide

Perform catalytic hydrogenation, under hydrogen at atmospheric pressure, at ambient temperature and overnight, on the compound prepared in Example 39, in the presence of palladium-on-carbon 10%. Remove the palladium and evaporate the filtrate under reduced pressure. Recrystallise the resulting precipitate from a mixture of methanol/water 90/10.

Melting point 170-173° C. (methanol/water)

### EXAMPLE 41

### 1-(4-Methoxybenzyl)-1H-pyrido[3,2-c][1,2,5]benzoxathiazepine 5,5-dioxide

The expected compound is obtained according to the procedure described in Example 1, starting from the compound prepared in Step B of Preparation C.

Melting point 162-163° C. (ethanol)

### EXAMPLE 42

### 1-[2-(4-Methoxyphenyl)ethyl]-1H-pyrido[3,2-c][1,2,5]benzoxathiazepine 5,5-dioxide

The expected compound is obtained according to the procedure described in Example 41, except that the 4-methoxybenzyl chloride is replaced by 4-methoxyphenylethyl methanesulphonate.

Melting point 115-116° C. (ethanol)

### EXAMPLE 43

### 1-[3-(4-Methoxyphenyl)propyl]-1H-pyrido[3,2-c][1,2,5]benzoxathiazepine 5,5-dioxide

The expected compound is obtained according to the procedure described in Example 41, except that the 4-methoxybenzyl chloride is replaced by 3-(4-methoxyphenyl)propyl methanesulphonate.

Melting point 99-100° C. (ethanol)

### EXAMPLE 44

### 1-[2-(3,4,5-Trimethoxyphenyl)ethyl]-1H-pyrido[3,2-c][1,2,5]benzoxathiazepine 5,5-dioxide

The expected compound is obtained according to the procedure described in Example 41, except that the 4-methoxybenzyl chloride is replaced by 2-(3,4,5-trimethoxyphenyl)ethyl methanesulphonate.

Melting point 165-166° C. (ethanol)

### EXAMPLE 45

### 1-[2-(1-Naphthyl)ethyl]-1H-pyrido[3,2-c][1,2,5]benzoxathiazepine 5,5-dioxide

The expected compound is obtained according to the procedure described in Example 41, except that the 4-methoxybenzyl chloride is replaced by [2-(1-naphthyl)ethyl]methanesulphonate.

Melting point 201-203° C. (ethanol)

### EXAMPLE 46

### 1-(2-[1,1′-Biphenyl]-4-ylethyl)-1H-pyrido[3,2-c][1,2,5]benzoxathiazepine 5,5-dioxide

The expected compound is obtained according to the procedure described in Example 41, except that the 4-methoxybenzyl chloride is replaced by [2-(4-biphenyl)ethyl]methanesulphonate.

Melting point 163-165° C. (ethanol)

### EXAMPLE 47

### 1-{2-[4-(Benzyloxy)phenyl]ethyl}-1H-pyrido[3,2-c][1,2,5]benzoxathiazepine 5,5-dioxide

The expected compound is obtained according to the procedure described in Example 41, except that the 4-methoxybenzyl chloride is replaced by 2-(4-benzyloxyphenyl)ethyl methanesulphonate.

Melting point 142° C. (ethanol)

### EXAMPLE 48

### 9-Chloro-1-[2-(4-methoxyphenyl)ethyl]-1H-pyrido[3,2-c][1,2,5]benzoxathiazepine 5,5-dioxide

The expected compound is obtained according to the procedure described in Example 41, replacing the 2-aminophenol in Step A of Preparation C by 2-amino-4-chlorophenol, and the 4-methoxybenzyl chloride is replaced by 4-methoxyphenylethyl methanesulphonate.

Melting point 126-127° C. (ethanol)

### EXAMPLE 49

### 9-Methyl-1-[2-(4-methoxyphenyl)ethyl]-1H-pyrido[3,2-c][1,2,5]benzoxathiazepine 5,5-dioxide

The expected compound is obtained according to the procedure described in Example 41, replacing the 2-aminophenol in Step A of Preparation C by 2-amino-4-methylphenol, and the 4-methoxybenzyl chloride is replaced by 4-methoxyphenylethyl methanesulphonate.

Melting point 114-115° C. (ethanol)

### EXAMPLE 50

### 9-Methoxy-1-[2-(4-methoxyphenyl)ethyl]-1H-pyrido[3,2-c][1,2,5]benzoxathiazepine 5,5-dioxide

The expected compound is obtained according to the procedure described in Example 41, replacing the 2-aminophenol in Step A of Preparation C by 2-amino-4-methoxyphenol, and the 4-methoxybenzyl chloride is replaced by 4-methoxyphenylethyl methanesulphonate.

Melting point 115-116° C. (ethanol)

### EXAMPLE 51

### 2-Chloro-1-[2-(4-methoxyphenyl)ethyl]-1H-pyrido[3,2-c][1,2,5]benzoxathiazepine 5,5-dioxide

The expected compound is obtained according to the procedure described in Example 41, replacing the 2-chloro-3-pyridinesulphochloride in Step A of Preparation C by 2,4-dichloro-3-pyridinesulphochloride, and the 4-methoxybenzyl chloride is replaced by 4-methoxyphenylethyl methanesulphonate.

Mass spectrum [M⁺]=416

### EXAMPLE 52

### 5-Methyl-1-[2-(3,4,5-trimethoxyphenyl)ethyl]-1,5-dihydropyrido[3,2-c][1,2,5]benzothiadiazepine 6,6-dioxide

The expected compound is obtained according to the procedure described in Example 15, except that the 4-methoxybenzyl chloride is replaced by 2-(3,4,5-trimethoxyphenyl)ethyl methanesulphonate.

Melting point 165-166° C. (ethanol)

### EXAMPLE 53

### 5-Methyl-1-[2-(4-N,N-dimethylaminoethoxyphenyl)ethyl]-1,5-dihydropyrido[3,2-c][1,2,5]benzothiadiazepine 6,6-dioxide

The expected compound is obtained according to the procedure described in Example 15, except that the 4-methoxybenzyl chloride is replaced by 2-(4-N,N-dimethylaminoethoxyphenyl)ethyl methanesulphonate.

Mass spectrum [M⁺]=452

### EXAMPLE 54

### 6-[(2-Methoxyethoxy)methyl]-1-[2-(4-methoxyphenyl)ethyl]-1,6-dihydropyrido[3,2-c][2,1,5]benzothiadiazepine 5,5-dioxide

The expected compound is obtained according to the procedure described in Example 2, replacing the azepine of Preparation A by that of Preparation E.

¹H NMR (solvent CDCl₃): δ (ppm): 3.10 (m,2H); 3.34 (s,3H); 3.52 (t,2H); 3.81 (s,3H); 3.95 (m,2H); 4.30 (m,2H); 4.90 (m,2H); 5.81 (dd,1H); 6.86 (d,2H); 7-7.15 (m,4H); 7.20-7.35 (m,3H); 7.85 (dd,1H).

### EXAMPLE 55

### 1-[2-(4-Methoxyphenyl)ethyl]-1,6-dihydropyrido[3,2-c][2,1,5]benzothiadiazepine 5,5-dioxide

Reflux a mixture of the compound of Example 54 (0.001 mol), 95° ethanol (10 ml) and 6N hydrochloric acid (10 ml) for 1 hour 30 minutes. After reaction, evaporate off as much of the ethanol as possible, dilute with water and add ethyl acetate. Neutralise with saturated sodium bicarbonate solution. Then re-acidify to pH 4-5 using acetic acid. Extract with ethyl acetate and wash with brine. Dry over sodium sulphate. Evaporate under reduced pressure and recrystallise from the appropriate solvent.

Melting point 174-175° C. (diisopropyl ether)

### EXAMPLE 56

### 4-[2-(5,5-Dioxido-1H-pyrido[3,2-c][1,2,5]benzoxathiazepin-1-yl)ethyl]phenol

Heat a mixture of the compound of Example 47 (0.0004 mol), hydrobromic acid (4 ml) and acetic acid (6 ml) at 35° C. for 5 days. Take up the mixture in ice and water. Add ethyl acetate and neutralise with sodium bicarbonate. Extract with ethyl acetate and wash with brine. Dry the organic phase over sodium sulphate. Filter and evaporate under reduced pressure. Recrystallise the precipitate from the appropriate solvent.

Melting point 58-61° C. (ethanol/water)

### EXAMPLE 57

### 1-[2-(2-Methoxyphenyl)ethyl]-1H-pyrido[3,2-c][1,2,5]benzoxathiazepine 5,5-dioxide

The expected compound is obtained according to the procedure described in Example 41, except that the 4-methoxybenzyl chloride is replaced by 2-(2-methoxyphenyl)ethyl methanesulphonate.

Melting point 138-140° C. (isopropanol/water)

### EXAMPLE 58

### 1-{2-[3-(Benzyloxy)-4-methoxyphenyl]ethyl}-1H-pyrido[3,2-c][1,2,5]benzoxathiazepine 5,5-dioxide

The expected compound is obtained according to the procedure described in Example 41, except that the 4-methoxybenzyl chloride is replaced by 2-[3-(benzyloxy)-4-methoxyphenyl]ethyl methanesulphonate.

¹H NMR (solvent CDCl₃): δ (ppm): 3.05 (t,2H); 3.89 (s,3H); 4.24 (t,2H); 5.14 (s,2H); 5.71 (dd,1H); 6.70-7.45 (m,13H); 7.83 (dd,1H).

### EXAMPLE 59

### 5-[2-(5,5-Dioxido-1H-pyrido[3,2-c][1,2,5]benzoxathiazepin-1-yl)ethyl]-2-methoxyphenol

The expected compound is obtained according to the procedure described in Example 56, replacing the compound of Example 47 as starting reagent by the compound of Example 58.

Melting point 158-160° C. (isopropanol/water)

### EXAMPLE 60

### 1-[2-(4-Methoxyphenyl)ethyl]pyrido[3,2-c][1,5]benzoxazepin-5(1H)-one

The expected compound is obtained according to the procedure described in Example 42, starting from the compound prepared in Step B of Preparation D.

### EXAMPLE 61

### 1-[2-(3-Hydroxy-4-methoxyphenyl)ethyl]pyrido[3,2-c][1,5]benzoxazepin-5(1H)-one

The expected compound is obtained according to the procedure described in Example 59, starting from the compound prepared in Step B of Preparation D.

### EXAMPLE 62

### 1-[2-(4-Methoxyphenyl)ethyl]-6-methyl-1,6-dihydro-5H-pyrido[2,3-b][1,5]benzodiazepin-5-one

The expected compound is obtained according to the procedure described in Example 42, starting from the compound prepared in Step B of Preparation F.

### EXAMPLE 62

### 1-[2-(3-Hydroxy-4-methoxyphenyl)ethyl]-6-methyl-1,6-dihydro-5H-pyrido[2,3-b][1,5]benzodiazepin-5-one

The expected compound is obtained according to the procedure described in Example 59, starting from the compound prepared in Step B of Preparation F.

### Pharmacological Study of Compounds of the Invention

### EXAMPLE A

### In Vitro Cytotoxicity

Five cell lines were used:
1 murine leukaemia, L1210,
1 non-small-cell human lung carcinoma, A549,
1 human epidermoid carcinoma, KB-3-1, and the corresponding resistant line, KB-A1, whose multi-drug resistance was induced using adriamycin (ADR),
1 human colon carcinoma, HT29.

The cells are cultured in RPMI 1640 complete culture medium comprising 10% foetal calf serum, 2 mM glutamine, 50 units/ml of penicillin, 50 μg/ml of streptomycin and 10 mM Hepes, pH 7.4. The cells are distributed on microplates and are exposed to the cytotoxic compounds. The cells are then incubated for 2 days (L1210) or 4 days (A549, KB-A1, KB-3-1, HT29). The number of viable cells is then quantified by a colorimetric assay, the Microculture Tetrazolium Assay (Cancer Res. 1987, 47, 936-942).

The results are expressed as IC₅₀, the concentration of cytotoxic agent that inhibits the proliferation of the treated cells by 50%. By way of example, the compound of Example 42 has the IC₅₀ values given in the Table below:

| | IC₅₀ nM | | | | |
|---|---|---|---|---|---|
| Test compounds | HT29 | L1210 | A549 | KB-3-1 | KB-A1 |
| Example 42 | 9.8 | 8.2 | 11.3 | 15.6 | 13.3 |

The compound of Example 42 is accordingly powerfully cytotoxic in those tumour lines. The resistant line KB-A1 is as sensitive as the sensitive line KB-3-1, which demonstrates that 42 is not recognised by P-glycoprotein, which is responsible for the multiple resistance to cytotoxic drugs.

Therefore, the compounds of the invention are, in addition, of value in the treatment of human tumours that are resistant to chemotherapy.

### EXAMPLE B

### Action on the Cell Cycle

L1210 cells are incubated for 21 hours at 37° C. in the presence of various concentrations of test compounds. The cells are then fixed using 70% ethanol (v/v), washed twice in PBS and incubated for 30 minutes at 20° C. in PBS that contains 100 μg/ml of RNAse and 50 μg/ml of propidium iodide. The results are expressed as a percentage of the cells that have accumulated in the G2+M phases after 21 hours compared with the control.

The compounds of the invention are powerful cytotoxic agents having selective action on the cell cycle. By way of example, the compound of Example 42 at a concentration of 25 nM causes 80-90% of the cells to accumulate in the G2+M phases after 21 hours (untreated cells: 20% in the G2+M phases).

### EXAMPLE C

### Pharmaceutical Composition

Formula for the preparation of 1000 tablets each containing 10 mg of active ingredient

| | |
|---|---|
| Compound of Example 42 | 10 g |
| Hydroxypropylcellulose | 2 g |
| Wheat starch | 10 g |
| Lactose | 100 g |
| Magnesium stearate | 3 g |
| Talc | 3 g |

1-20. (canceled)

21- A compound selected from those of formula (I): 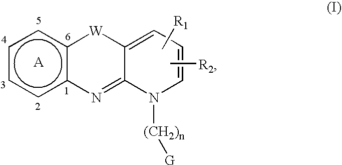
wherein: 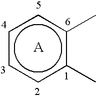
represents a benzo or pyrido group, optionally fused in the 2-3, 3-4 or 4-5 position to a phenyl, (C₄-C₈) cycloalkyl or heterocyclic group, it being understood that the nitrogen of the pyrido group occupies any of positions 2 to 5 in the ring, which may be optionally substituted by one or more identical or different atoms or groups selected from halogen, hydroxy, linear or branched (C₁-C₆)alkyl, linear or branched (C₁-C₆)alkoxy, linear or branched (C₁-C₆)trihaloalkyl, amino (optionally substituted at the nitrogen atom by one or two linear or branched (C₁-C₆)alkyl groups), nitro, linear or branched (C₁-C₆)acyl and (C₁-C₂)alkylenedioxy,
W represents X—Y or Y—X, wherein:
X represents 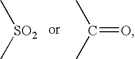
and Y represents oxygen or N—R₃, wherein R₃ represents hydrogen, linear or branched (C₁-C₆)alkyl, linear or branched aryl(C₁-C₆)alkyl, -Alk-Z-R or -Alk-Z-Alk′-Z′-R, wherein Alk and Alk′, which may be the same or different, each independently represent linear or branched (C₁-C₆)alkylene or linear or branched (C₂-C₆)alkenylene, Z and Z′, which may be the same or different, each independently represent oxygen, sulphur or —N(R′)— and R and R′, which may be the same or different, each independently represent linear or branched (C₁-C₆)alkyl,
n represents zero or an integer from 1 to 6,
G represents hydrogen, aryl or heteroaryl,
R₁ and R₂, which may be the same or different, each independently represent hydrogen, halogen, hydroxy, linear or branched (C₁-C₆)alkyl, linear or branched (C₁-C₆)alkoxy, linear or branched (C₁-C₆)trihaloalkyl, amino (optionally substituted at the nitrogen by one or two linear or branched (C₁-C₆)alkyl), nitro, linear or branched (C₁-C₆)acyl, or a (C₁-C₂)alkylenedioxy group
its enantiomers, diastereoisomers, and addition salts thereof with a pharmaceutically acceptable acid or base,
it being understood that:
n is other than zero when G represents hydrogen,
when G represents hydrogen and Y represents N—R₃, then R₃ represents hydrogen, linear or branched (C₂-C₆)alkyl or aryl-(C₁-C₆)alkyl wherein alkyl is linear or branched,
when G represents hydrogen and W represents one of the two NR₃C(O) groups wherein R₃ represents ethyl or benzyl, n is other than 1, 2 or 3,
the compounds of formula (I) are other than
1-benzyl-5,10-dimethyl-1,5-dihydro-6H-pyrido[2,3-b][1,4]benzodiazepin-6-one, ethyl 1,2-dimethyl-5-oxo-5,6-dihydro-1H-pyrido[2,3-b][1,5]benzodiazepine 3-carboxylate,
3-acetyl-1-ethyl-2-methyl-1,6-dihydro-5H-pyrido[2,3-b][1,5]benzodiazepin-5-one, 2-amino-1-methyl-5-oxo-5,6-dihydro-1H-pyrido[2,3-b][1,5]benzodiazepine-3-carbonitrile and
ethyl 2-amino-1-methyl-5-oxo-5,6-dihydro-1H-pyrido[2,3-b][1,5]benzodiazepine 3-carboxylate,
“aryl” means phenyl, biphenyl, naphthyl, or tetrahydronaphthyl, each of those groups being optionally substituted by one, two or three identical or different atoms or groups selected from halogen, linear or branched (C₁-C₆)alkyl, hydroxy, linear or branched (C₁-C₆)alkoxy, linear or branched (C₁-C₆)trihaloalkyl, amino (optionally substituted at the nitrogen atom by one or two linear or branched (C₁-C₆)alkyl groups), nitro, linear or branched (C₁-C₆)acyl, linear or branched (C₁-C₆)alkylcarbonylamino, (C₁-C₂)alkylenedioxy, phenyloxy, benzyloxy, linear or branched amino-(C₁-C₆)alkoxy, linear or branched (C₁-C₆)alkylamino-(C₁-C₆)alkoxy and linear or branched di(C₁-C₆)alkylamino-(C₁-C₆)alkoxy,
“heteroaryl” means a mono- or bi-cyclic, aromatic, 5- to 12-membered group having one, two or three hetero atoms selected from oxygen, nitrogen and sulphur, wherein the heteroaryl may be optionally substituted by one or more identical or different atoms or groups selected from halogen, linear or branched (C₁-C₆)alkyl, hydroxy, linear or branched (C₁-C₆)alkoxy, linear or branched (C₁-C₆)trihaloalkyl, amino (optionally substituted by one or more linear or branched (C₁-C₆)alkyl groups), nitro, linear or branched (C₁-C₆)acyl, linear or branched (C₁-C₆)alkylcarbonylamino, linear or branched (C₁-C₂)alkylenedioxy, phenyloxy, benzyloxy, linear or branched amino-(C₁-C₆)alkoxy, linear or branched (C₁-C₆)alkylamino-(C₁-C₆)alkoxy and linear or branched di(C₁-C₆)alkylamino-(C₁-C₆)alkoxy,
“group” 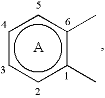
optionally fused in the 2-3, 3-4 or 4-5 position to a phenyl, (C₄-C₈)cycloalkyl or heterocyclic group, means that the benzo or pyrido group is optionally fused to a phenyl, (C₄-C₈)cycloalkyl or heterocyclic group in the position 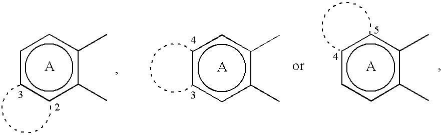
it being understood that, when 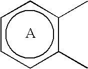
represents a pyrido group, the nitrogen atom is not a point of attachment to the fused ring,
“alkylene” means bivalent radical of saturated hydrocarbon chain,
“alkenylene” means bivalent radical of hydrocarbon chain having from 1 to 3 double bonds,
“(C₄-C₈)cycloalkyl” means cyclobutane, cyclopentane, cyclohexane, cycloheptane or cyclooctane, and
“heterocyclic” means a saturated or unsaturated, 5- to 7-membered, monocyclic group having from one to three hetero atoms selected from nitrogen, oxygen and sulphur.

22- A compound of claim 21, wherein
X represents 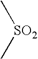
and Y represents N—R₃,
its enantiomers and diastereoisomers, and addition salts thereof with a pharmaceutically acceptable acid or base.

23- A compound of claim 21, wherein
X represents 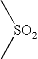
and Y represents O,
its enantiomers and diastereoisomers, and addition salts thereof with a pharmaceutically acceptable acid or base.

24- A compound of claim 21, wherein
X represents 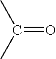
and Y represents N—R₃,
its enantiomers and diastereoisomers, and addition salts thereof with a pharmaceutically acceptable acid or base.

25- A compound of claim 21, wherein
X represents 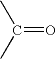
and Y represents O,
its enantiomers and diastereoisomers, and addition salts thereof with a pharmaceutically acceptable acid or base.

26- A compound of claim 21, wherein G represents aryl or heteroaryl, its enantiomers and diastereoisomers, and addition salts thereof with a pharmaceutically acceptable acid or base.

27- A compound of claim 26, wherein G represents phenyl substituted by one, two or three groups selected from linear or branched (C₁-C₆)alkoxy, benzyloxy and hydroxy, its enantiomers and diastereoisomers, and addition salts thereof with a pharmaceutically acceptable acid or base.

28- A compound of claim 21, wherein R₃ represents linear or branched (C₁-C₆)alkyl, its enantiomers and diastereoisomers, and addition salts thereof with a pharmaceutically acceptable acid or base.

29- A compound of claim 21, wherein R₃ represents hydrogen or linear or branched aryl(C₁-C₆)alkyl, its enantiomers and diastereoisomers, and addition salts thereof with a pharmaceutically acceptable acid or base.

30- A compound of claim 21, wherein 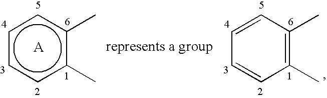
optionally substituted by 1, 2 or 3 identical or different atoms or groups selected from halogen, hydroxy, linear or branched (C₁-C₆)alkyl, linear or branched (C₁-C₆)alkoxy, linear or branched (C₁-C₆)trihaloalkyl, amino (optionally substituted at the nitrogen atom by one or two linear or branched (C₁-C₆)alkyl groups), nitro, linear or branched (C₁-C₆)acyl and (C₁-C₂)alkylenedioxy, its enantiomers and diastereoisomers, and addition salts thereof with a pharmaceutically acceptable acid or base.

31- A compound of claim 30, wherein the substituents are located in the 3- or 4-position of the group 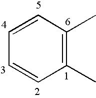
and are selected from halogen, linear or branched (C₁-C₆)alkyl, linear or branched (C₁-C₆)alkoxy and linear or branched (C₁-C₆)trihaloalkyl, its enantiomers and diastereoisomers, and addition salts thereof with a pharmaceutically acceptable acid or base.

32- A compound of claim 21, wherein R₁ and R₂, which may be the same or different, each independently represent hydrogen, halogen, linear or branched (C₁-C₆)alkyl, linear or branched (C₁-C₆)alkoxy or linear or branched (C₁-C₆)trihaloalkyl, its enantiomers and diastereoisomers, and addition salts thereof with a pharmaceutically acceptable acid or base.

33- A compound of claim 21, which is 6-[(2-methoxyethoxy)methyl]-1-[2-(4-methoxyphenyl)ethyl]-1,6-dihydropyrido-[3,2-c][2,1,5]benzothiadiazepine-5,5-dioxide, and addition salts thereof with a pharmaceutically acceptable acid.

34- A compound of claim 21, which is 1-[2-(4-methoxyphenyl)ethyl]-1,6-dihydropyrido[3,2-c][2,1,5]benzothiadiazepine 5,5-dioxide, and addition salts thereof with a pharmaceutically acceptable acid.

35- A compound of claim 21, which is selected from:
1-[2-(4-methoxyphenyl)ethyl]-6-methyl-1,6-dihydropyrido[3,2-c][2,1,5]benzothiadiazepine 5,5-dioxide,
1-[2-(4-methoxyphenyl)ethyl]-5-methyl-1,5-dihydropyrido[3,2-c][1,2,5]benzothiadiazepine 6,6-dioxide,
and addition salts thereof with a pharmaceutically acceptable acid.

36- A compound of claim 21, which is selected from:
1-[2-(4-methoxyphenyl)ethyl]-1H-pyrido[3,2-c][1,2,5]benzoxathiazepine 5,5-dioxide,
4-[2-(5,5-dioxido-1H-pyrido[3,2-c][1,2,5]benzoxathiazepin-1-yl)ethyl]phenol,
1-[2-(2-methoxyphenyl)ethyl]-1H-pyrido[3,2-c][1,2,5]benzoxathiazepine 5,5-dioxide,
1-{2-[3-(benzyloxy)-4-methoxyphenyl]ethyl}-1H-pyrido[3,2-c][1,2,5]benzoxathiazepine 5,5-dioxide,
5-[2-(5,5-dioxido-1H-pyrido[3,2-c][1,2,5]benzoxathiazepin-1-yl)ethyl]-2-methoxyphenol,
and addition salts thereof with a pharmaceutically acceptable acid.

37- A pharmaceutical composition useful for the treatment of cancerous diseases, comprising as active principle an effective amount of a compound of claim 21, together with one or more pharmaceutically acceptable excipients or vehicles.

38- A pharmaceutical composition useful for the treatment of leukaemia, adenocarcinoma and carcinoma, comprising as active principle an effective amount of a compound of claim 21, together with one or more pharmaceutically acceptable excipients or vehicles.

39- A method for treating a living animal body, including a human, afflicted with a condition associated with cancerous diseases comprising the step of administering to the living animal body, including a human, an amount of a compound of claim 21 which is effective for alleviation of the condition.

40- A method for treating an living animal body, including a human, afflicted with a condition associated with leukaemia, adenocarcinoma and carcinoma comprising the step of administering to the living animal body, including a human, an amount of a compound of claim 21, which is effective for alleviation of the condition.
